(19) United States
(12) Patent Application Publication
(10) Pub. No.: US 2026/0128125 A1
(43) Pub. Date: May 7, 2026

(54) METHOD FOR INDIVIDUAL-SPECIFIC NEIGHBORHOOD-BASED POLYGENIC RISK MODELING, DEBIASED FROM ANCESTRY EFFECTS, FOR IMPROVED DISEASE RISK PREDICTION

(71) Applicant: ALLELICA S.R.L., Roma (IT)

(72) Inventors: Paolo DI DOMENICO, Roma (IT); Giordano BOTTÀ, Roma (IT)

(21) Appl. No.: 19/382,099

(22) Filed: Nov. 6, 2025

Related U.S. Application Data
(60) Provisional application No. 63/717,680, filed on Nov. 7, 2024.

(51) Int. Cl. G16B 20/40; G16B 40/20; G16H 50/30

(52) U.S. Cl. CPC G16B 20/40; G16B 40/20; G16H 50/30

(57) ABSTRACT

A computer-implemented method for calculating an individual's tailored Polygenic Risk Score is based on known genetic information. A dataset is provided related to a reference panel including genetically characterized individuals with known disease status and diversified global ancestry. An individual-specific genetic reference group of individuals is selected as a subset from the reference panel. Genetic distances of the individual from each of the reference panel individuals are computed; each being the individual's genetic distance from a respective reference panel individual. Individuals of the individual-specific genetic reference group based on the individual's computed genetic distances are selected. The individual's basic Polygenic Risk Score for a disease is calculated to provide the individual's disease risk prediction. An ancestry-based background PRS contribution is determined. The ancestry contribution is removed from the individual's calculated basic Polygenic Risk Score to obtain the individual's tailored Polygenic Risk Score and provide a disease risk prediction.

### CROSS REFERENCE TO RELATED APPLICATION

This application claims priority to and benefit of U.S. Provisional Application No. 63/717,680 filed Nov. 7, 2024, the contents of which are incorporated by reference in their entirety.

### FIELD OF APPLICATION

The present disclosure relates to a computer-implemented method for individual-specific neighborhood-based polygenic risk modeling, debiased from ancestry effects, for improved disease risk prediction.

The general technical field of the present disclosure is therefore the field of predictive methods, performed by means of electronic computation, used in the medical field to support predictive prognoses.

More specifically, the present disclosure refers to a computer-implemented method for calculating a tailored Polygenic Risk Score, PRS_{T}, specific to an individual, based on known genetic information of the individual, and debiased from ancestry-related effects.

Moreover, the present disclosure also refers to a computer-implemented method for predicting the risk of a disease for an individual, based on the abovementioned method for calculating a tailored individual-specific Polygenic Risk Score, PRS_{T}.

### PRIOR ART

Polygenic Risk Scores (PRSs) are pivotal in predicting complex disease risks, and are broadly used for predictive methods, in order to support predictive prognoses taking into account genetic factors.

However, it is well known that genetic differences across ancestry groups critically undermine the accuracy of PRS-based methods.

The Polygenic Risk Scores, PRS, although it is a deterministic parameter, requires to be adjusted taking into account of ancestry-related effects.

In other terms, a customized PRS, tailored to a specific individual, must be debiased from an ancestry-related background contribution.

Conventional approaches attempt to correct for this issue by using broad, pre-defined population groups such as continental ancestry bins (e.g., African, European, East Asian).

However, these known approaches are often overly simplistic and do not capture the fine-scale genetic variation that influences risk prediction.

Consequently, individuals from non-Europeans or admixed populations are often misclassified, or their risk is inaccurately estimated, leading to biased predictions, reduced clinical utility, and potential health disparities (as explained, for example, in the paper “Polygenic scoring accuracy varies across the genetic ancestry continuum”, Yi Ding et al. Nature, 2023).

The abovementioned conventional approaches to genetic risk prediction are fundamentally label-based: they assign individuals to predefined ancestry groups—often, but not exclusively, continental.

These label-based models (see for example “Whole-Genome Sequencing to Characterize Monogenic and Polygenic Contributions in Patients Hospitalized With Early-Onset Myocardial Infarction”—Kera et al. Circulation 2019) typically rely on:
linear principal components (PCs), i.e., models having PCs as predictors and PRS as predicted variable, used in order to adjust for broad-scale population structure;
residual risk modeling, performed within each ancestry label to account for within-group variation.

However, the act of labeling imposes rigid boundaries on what is inherently a continuous, high-dimensional space of genetic variation. This simplification can obscure fine-scale differences and introduce bias, particularly for individuals whose ancestry does not fit neatly into discrete categories.

On the whole, the abovementioned known models provide rough approximate results, with a “coarse” granularity.

Therefore, there is still a strong need to devise methods for calculating a tailored Polygenic Risk Score, PRS_{T}, specific to an individual, based on known genetic information of the individual, and debiased from ancestry-related effects in a more effective manner than the currently known and conventional solutions.

### SUMMARY

It is the object of the present disclosure to provide a method for calculating a tailored Polygenic Risk Score, PRS_{T}, specific to an individual, based on known genetic information of the individual, and debiased from ancestry-related effects, which allows to obviate at least partially the drawbacks mentioned above with reference to the known art, and to meet the aforementioned needs particularly felt in the technical field considered.

### BRIEF DESCRIPTION OF THE DRAWINGS

Further features and advantages of the method according to this disclosure will become apparent from the following description of preferred embodiments, given by way of non-limiting example, with reference to the accompanying drawings, in which:

FIG. 1 is a flow diagram of an embodiment of a method for generating an individual-specific tailored Polygenic Risk Score according to the present disclosure;

FIG. 2 is a flow diagram representing the additional steps that are carried out, in addition and downstream the steps described in FIG. 1, in an embodiment of a method for predicting the risk of a disease for an individual according to the present disclosure;

### DETAILED DESCRIPTION

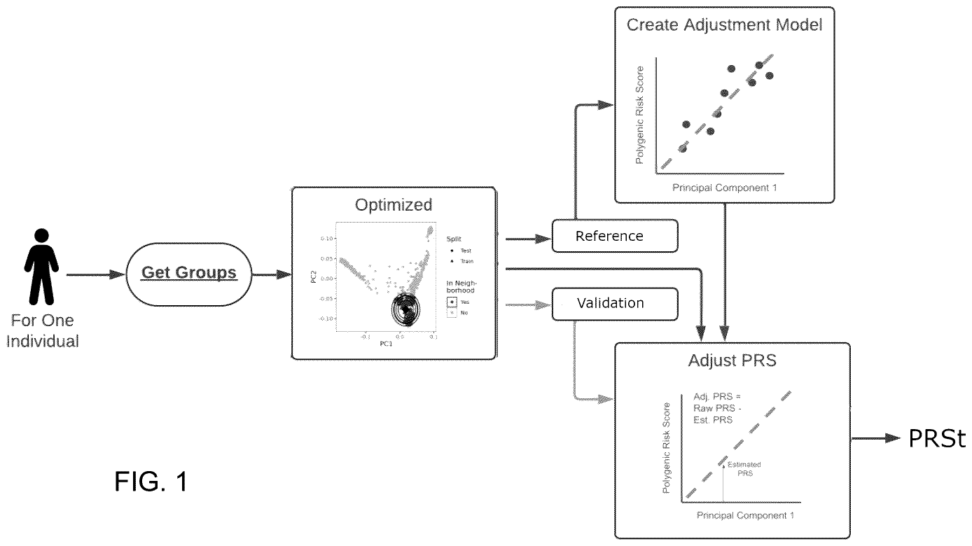

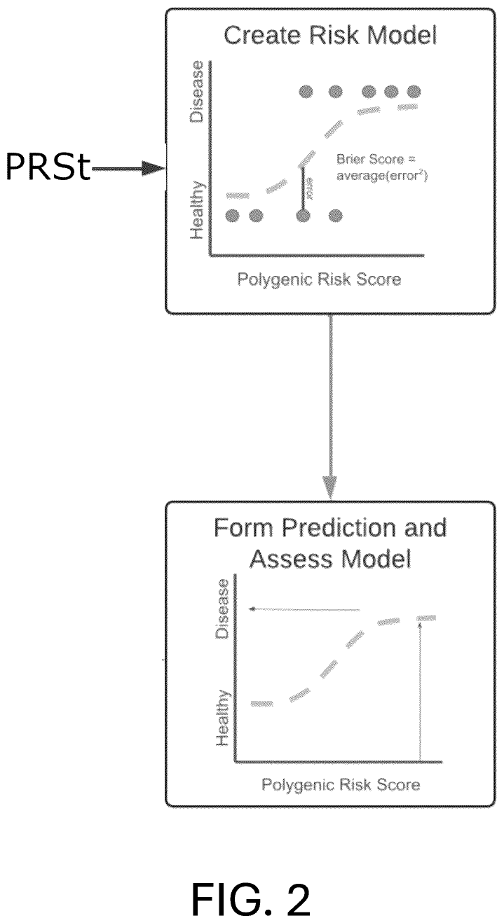

Referring to the FIGS. 1-2, a computer-implemented method for calculating a tailored Polygenic Risk Score, PRS_{T}, specific to an individual, based on known genetic information of the individual, is described.

The method firstly comprises the step of providing a dataset related to a reference panel including genetically characterized individuals with known disease status and diversified global ancestry.

Then, the method provides identifying an individual-specific genetic reference group of individuals selected as a subset from the aforesaid reference panel. Such a step of identifying comprises computing a plurality of genetic distances of the individual from each of the individuals of the reference panel; each genetic distance is the genetic distance of the individual from a respective one of the individuals of the reference panel.

The method further comprises a step of selecting the individuals to be comprised in the individual-specific genetic reference group of individuals based on the computed genetic distances of the individual from all the individuals of the reference panel.

Then, the method provides a step of calculating a basic Polygenic Risk Score, PRS₀, of the individual with respect to a disease, suitable to provide a risk prediction for the individual with respect to said disease.

The method further comprises the steps of determining a background PRS contribution due to ancestry based on the aforesaid individual-specific genetic reference group of individuals, and finally debiasing the calculated basic Polygenic Risk Score, PRS₀, of the individual from ancestry-related effects, by removing the determined background PRS contribution due to ancestry, to obtain the tailored Polygenic Risk Score, PRS_{T}, specific to the individual, suitable to provide a risk prediction for said individual with respect to said disease.

According to an embodiment of the method, the abovementioned step of computing a genetic distance of the individual from all the individuals of the reference panel is carried out in a multi-dimensional data space representing genetic information.

In this case, the aforesaid individual-specific genetic reference group of individuals is a neighborhood around the point representing the individual in the multi-dimensional genetic information data space.

According to an implementation option of said embodiment, the step of computing a plurality of genetic distances of the individual from each of the individuals of the reference panel is carried out in the space of the principal component, PC, coordinates, representing genetic information. In this case, the individual-specific genetic reference group of individuals is a neighborhood around the point representing the individual in the space of the principal component, PC, coordinates.

According to an implementation option of said embodiment, the step of computing a plurality of genetic distances of the individual from each of the individuals of the reference panel is carried out by using identity by descent, IBD, information.

According to an embodiment of the method, the abovementioned step of identifying an individual-specific neighborhood comprises the following sub-steps:
selecting a first set of individuals as components of an initial candidate neighborhood;
evaluating the initial candidate neighborhood based on a predefined criterion for assessing the prediction efficacy of the tailored Polygenic Risk Score, PRS_{T}, calculated on the basis of said initial candidate neighborhood;
iteratively modifying the initial candidate neighborhood to obtain further candidate neighborhoods, and evaluating the further candidate neighborhoods based on said predefined criterion, in order to obtain an optimized individual-specific neighborhood.

According to an implementation option of said embodiment, the step of evaluating a candidate neighborhood comprises fitting a regression model with Polygenic Risk Score, PRS, as the predictor, disease phenotype as the outcome, and covariates representing the individual's conditions or characteristics.

In this case, the regression model represents the abovementioned predefined criterion for assessing the prediction efficacy of the tailored Polygenic Risk Score.

The regression model is, for example, a logistic regression model, per se known.

In an implementation option, the covariates representing the individual's conditions or characteristics comprises, for example, age and sex.

According to an implementation option of said embodiment, the step of iteratively modifying the initial candidate neighborhood to obtain further candidate neighborhoods comprises performing a stochastic optimization procedure to explore forward and backward inclusion and exclusion steps, in order to determine the optimal neighborhood size and composition for the considered individual, thus obtaining the individual-specific neighborhood to be used.

The iteration provides that, once dynamically generated and evaluated a candidate neighborhood, such a candidate neighborhood is selected as the provisional best candidate neighborhood if it provides the best performances, according to the predefined evaluation criterion, among the candidate neighborhoods evaluated at the moment, otherwise it is discarded, and the iteration continues.

According to a method embodiment, the optimal neighborhood size for polygenic risk score calculation is determined based on several factors. e.g., disease prevalence, trait heritability, and individual ancestry, making it both trait- and individual-specific. This method automatically accounts for these factors to maximize risk calibration.

According to an embodiment of the method, the abovementioned step of calculating a basic Polygenic Risk Score, PRS₀, of the individual with respect to a disease, suitable to provide a risk prediction for said individual with respect to said disease, is carried out by means of known procedures, wherein the calculation depends on the kind of disease to be predicted.

As examples of calculation of PRS, the patent application US 2023/0111182 A1, in the name of the Applicant, describes a calculation of a PRS for predicting the onset of menopause; the patent application US 2023/0260659 A1, in the name of the Applicant, describes a calculation of a PRS for predicting a cardiovascular disease.

According to an embodiment of the method, the abovementioned step of determining a background PRS contribution due to ancestry based on said individual-specific genetic reference group of individuals comprises performing a regression of the Polygenic Risk Score values of the members of said selected individual-specific genetic reference group of individuals against their principal component, PC, coordinates, thus capturing global ancestry structure; and considering the fitted component of said regression as representative of the background PRS contribution due to ancestry.

According to an embodiment of the method, the abovementioned step of debiasing the calculated basic Polygenic Risk Score, PRS₀, of the individual from ancestry-related effects comprises removing the determined background PRS contribution due to ancestry, by subtracting the calculated background PRS contribution from the basic Polygenic Risk Score, PRS_{T}, to obtain the tailored Polygenic Risk Score, PRS_{T}.

According to an embodiment the method further comprises, after the step of identifying an individual-specific genetic reference group of individuals, a step of validating the identified individual-specific genetic reference group of individuals.

This validation is carried out by evaluating the prediction accuracy of the tailored Polygenic Risk Score, PRS_{T}, calculated on the basis of the identified individual-specific genetic reference group of individuals and comparing it with the prediction accuracy of a standard Polygenic Risk Score debiased from ancestry-related effects on the basis of a labelling-based continental or geographical ancestry model which assigns the individual to a predefined continental/geographical-based ancestry group.

The validation mentioned above is a validation performed on a single tailored Polygenic Risk Score, PRS_{T}, calculated for a single individual, to check and possibly confirm that the single tailored Polygenic Risk Score, PRS_{T} provides better prediction than, at least, a Polygenic Risk Score debiased from ancestry effects by using label-based prior art procedures.

According to an implementation option of the method, this validation step is useful in view of the preparation of a predictive disease risk information to be provided to the individual, e.g., a patient.

According to an embodiment, the method provides another kind of validation, more general, referred to a general ancestry grouping model validation.

More specifically, in this case, the method further comprises, as a first preliminary step, a step of general ancestry grouping model validation comprising the following sub-steps:
performing the step of identifying an individual-specific genetic reference group of individuals for each of the individuals of said reference panel;
for each of the identified individual-specific genetic reference groups, evaluating the prediction accuracy of the respective tailored Polygenic Risk Score, PRS_{Ti}, and comparing it with the prediction accuracy of a standard Polygenic Risk Score debiased from
ancestry-related effects on the basis of a labelling-based continental or geographical ancestry model which assigns the individual to a predefined continental/geographical-based ancestry group;
validating as a usable ancestry grouping model a model providing better performances than the labelling-based continental or geographical ancestry model.

According to an implementation option of the abovementioned embodiment, the step of validating comprises counting the proportion of cases (i.e., prediction of disease) for which the neighborhood-based ancestry grouping model yields more accurate predictions, then counting the number of controls (i.e., prediction of no disease) for whom the neighborhood-based ancestry grouping model yields more accurate predictions, and finally validating the neighborhood-based ancestry grouping model only if both the proportion of improved predictions in cases and the number of improved predictions in controls exceed a predefined threshold.

According to an implementation example, said predefined threshold is 50 percent.

A computer-implemented method for predicting the risk of a disease for an individual, based on generic information of the individual, also comprised in this disclosure, is here described.

This method comprises performing a computer-implemented method for calculating a tailored Polygenic Risk Score, PRS_{T}, specific to an individual, according to any of the embodiments described above, and then predicting the risk of the disease for the individual based on the calculated tailored Polygenic Risk Score, PRS_{T}, specific to the individual and debiased from ancestry-related effects.

With reference to FIGS. 1-2, some further details on a specific embodiment of the method according to the present disclosure is provided here below, as an example provided for not limiting illustration purposes.

According to this embodiment, the method provides the following actions:
for each focal individual, computing genetic distance—using, for example, either principal component (PC) coordinates or identity by descent (IBD)—to all individuals in a reference panel, including genetically characterized individuals with known disease status and maximized global ancestral representation;
initializing a neighborhood using individuals from the reference panel, then iteratively evaluating different neighborhood sizes and compositions;
for each candidate neighborhood, fitting a regression model (e.g., logistic) with PRS as the predictor, disease phenotype as the outcome, and covariates such as age and sex;
performing a procedure (e.g., stochastic optimization) to explore forward and backward inclusion and exclusion steps, in order to determine the optimal neighborhood size and composition for each focal individual;
evaluating each of the obtained models by comparing its prediction accuracy to that of the known continental/geographical partition model, by counting the proportion of cases for which the neighborhood model yields more accurate predictions and counting the number of controls for whom the neighborhood model yields more accurate predictions;
retaining the neighborhood model only if both the proportion of improved predictions in cases and the number of improved predictions in controls exceed 50 percent;
if no model meets this criterion, fall back to the continental model for prediction;
for the final selected model, compute odds ratio per standard deviation, AUROC, and calculate adjusted PRS, PRS percentile, and overall model prediction.

FIG. 1 is a flow diagram of an embodiment of the method for generating an individual-specific tailored Polygenic Risk Score (PRSₜ). In this embodiment, the process begins with the provision of a reference panel comprising genetically characterized individuals with known disease status. These individuals define a multidimensional genetic space in which the individual of interest is embedded. An individual-specific neighborhood is constructed based on genetic proximity within this space. Within the neighborhood, statistical models are fitted using the PRS as predictor and disease status as outcome.

The grouping in optimized neighborhood of the individuals of the reference panel is shown in the diagram named “Optimized”, in FIG. 1, representing a bidimensional projection of the abovementioned multi-dimensional data space representing genetic information, wherein the variables in the axes are the principal components PC1, PC2. In FIG. 1, each colored point represents one individual of the reference panel, and the points corresponding to the individuals selected for the neighborhood are highlighted.

FIG. 2 is a flow diagram illustrating the use of the tailored Polygenic Risk Score (PRSt) within the optimal individual-specific model. Once PRSt is computed, it is applied to the final model fitted within the individual's genetically matched neighborhood. The model returns a personalized prediction of the individual's risk of developing the disease.

As a non-limiting example, provided for the mere purpose of further illustrating the present disclosure, it is noted that the Applicant has evaluated the performances of the above disclosed neighborhood-based method on 250 individuals from the MESA cohort across three diseases, i.e., in this example, Coronary Artery Disease (CAD), Atrial Fibrillation (AF), Type 2 Diabetes (T2D), using UK Biobank-based risk models.

The “UK BioBank” database (https://www.ukbiobank.ac.uk/) contains the genetic information of over 500,000 volunteers, providing clinical information.

Compared to the prior art continental/geographic partition ancestry approach, the neighborhood model, disclosed above, showed improved or equivalent performance in 95% of controls (no-disease) and 83% of cases (with disease) individuals. On average, selected models demonstrated a 74% higher odds ratio per standard deviation and a 10% higher AUROC, indicating superior predictive performance.

As can be seen, the objects of the present disclosure, as previously indicated, are fully achieved by the method described above, by virtue of the features disclosed above in detail.

The approach used in this disclosure to debias the PRS value from ancestry-related effects, in contrast with the known prior art solution, is explicitly label-free.

In other terms, it does not assign individuals to ancestry bins, but instead models genetic relatedness in a continuous manner, more accurately reflecting the underlying structure of human genetic diversity.

The solution of the present disclosure proposes a neighborhood-based algorithm that dynamically identifies an individual-specific genetic reference group, i.e., “neighborhood”—from a broader dataset. This group is composed of individuals who are most genetically similar to the target individual (whose tailored PRS has to be calculated), based on a high-resolution comparison of genome-wide variation.

Key features of the present disclosure include dynamic grouping (i.e., the PRS model of each individual is validated on a tailored cohort, not based on a fixed ancestry label); improved calibration and accuracy (statistical analyses show significantly better model calibration, Brier skill scores, and reduced prediction error compared to already established methods); equitable risk modeling, in that the method consistently improves performance across ancestries.

This label-free, individualized grouping strategy leverages genomic similarity rather than socio-geographic classification, representing a paradigm shift in how genetic risk is quantified and personalized, this enabling, in principle, the creation of what could be called “8 billion risk models”, a unique one for every human being.

In order to meet contingent needs, those skilled in the art may make modifications and adaptations to the embodiments of the method described above and can replace elements with others which are functionally equivalent without departing from the scope of the following claims. All the features described above as belonging to one possible embodiment may be implemented irrespective of the other embodiments described.

1. A computer-implemented method for calculating a tailored Polygenic Risk Score (PRS_{T}), specific to an individual, based on known genetic information of the individual, the method comprising the steps of:
providing a dataset related to a reference panel including genetically characterized individuals with known disease status and diversified global ancestry;
identifying an individual-specific genetic reference group of individuals selected as a subset from said reference panel, wherein said step of identifying comprises:
computing a plurality of genetic distances of the individual from each of the individuals of the reference panel, wherein each genetic distance is a genetic distance of the individual from a respective one of the individuals of the reference panel;
selecting the individuals to be comprised in the individual-specific genetic reference group of individuals based on the computed genetic distances of the individual from all the individuals of the reference panel;
calculating a basic Polygenic Risk Score (PRS₀), of the individual with respect to a disease, to provide a risk prediction for said individual with respect to said disease;
determining a background Polygenic Risk Score (PRS) contribution due to ancestry based on said individual-specific genetic reference group of individuals;
debiasing the calculated basic Polygenic Risk Score, PRS₀, of the individual from ancestry-related effects, by removing the determined background Polygenic Risk Score (PRS) contribution due to ancestry, to obtain the tailored Polygenic Risk Score (PRS_{T}), specific to the individual, and provide an improved risk prediction for said individual with respect to said disease.

2. The computer-implemented method according to claim 1, wherein said step of computing a plurality of genetic distances of the individual from each of the individuals of the reference panel is performed in a multi-dimensional data space representing genetic information, and wherein said individual-specific genetic reference group of individuals is a neighborhood around a point representing the individual in said multi-dimensional genetic information data space.

3. The computer-implemented method according to claim 2, wherein said step of computing a plurality of genetic distances of the individual from each of the individuals of the reference panel is performed in the space of principal component (PC) coordinates, representing genetic information, and wherein said individual-specific genetic reference group of individuals is a neighborhood around the point representing the individual in the space of the principal component (PC) coordinates.

4. The computer-implemented method according to claim 2, wherein said step of computing a genetic distance of the individual from all the individuals of the reference panel is performed by using identity by descent, IBD, information.

5. The computer-implemented method according to claim 2, wherein the step of identifying an individual-specific neighborhood comprises:
selecting a first set of individuals as components of an initial candidate neighborhood;
evaluating an initial candidate neighborhood based on a predefined criterion for assessing the prediction efficacy of the tailored Polygenic Risk Score (PRS_{T}) calculated based on said initial candidate neighborhood;
iteratively modifying the initial candidate neighborhood to obtain further candidate neighborhoods, and evaluating the further candidate neighborhoods based on said predefined criterion, to obtain an optimized individual-specific neighborhood.

6. The computer-implemented method according to claim 5, wherein the step of evaluating a candidate neighborhood comprises fitting a regression model with Polygenic Risk Score (PRS) as a predictor, disease phenotype as an outcome, and covariates representing the individual's conditions or characteristics.

7. The computer-implemented method according to claim 5, wherein the step of iteratively modifying the initial candidate neighborhood to obtain further candidate neighborhoods comprises:
performing a stochastic optimization procedure to explore forward and backward inclusion and exclusion steps, in order to determine the optimal neighborhood size and composition for the considered individual, thus obtaining the individual-specific neighborhood to be used.

8. The computer-implemented method according to claim 1, wherein the step of determining a background PRS contribution due to ancestry based on said individual-specific genetic reference group of individuals comprises:
performing a regression of the Polygenic Risk Score values of the members of said selected individual-specific genetic reference group of individuals against principal component (PC) coordinates, thus capturing global ancestry structure;
considering a fitted component of said regression as representative of the background PRS contribution due to ancestry.

9. The computer-implemented method according to claim 1, wherein the step of debiasing the calculated basic Polygenic Risk Score (PRS₀) of the individual from ancestry-related effects comprises removing the determined background PRS contribution due to ancestry, by subtracting the calculated background PRS contribution from the basic Polygenic Risk Score (PRS_{T}) to obtain the tailored Polygenic Risk Score (PRS_{T}).

10. The computer-implemented method according to claim 1, further comprising, after the step of identifying an individual-specific genetic reference group of individuals, a step of validating the identified individual-specific genetic reference group of individuals, by evaluating prediction accuracy of the tailored Polygenic Risk Score (PRS_{T}) calculated based on the identified individual-specific genetic reference group of individuals, and comparing the tailored Polygenic Risk Score (PRS_{T}) with the prediction accuracy of a standard Polygenic Risk Score debiased from ancestry-related effects based on a labelling-based continental ancestry model or geographical ancestry model which assigns the individual to a predefined continental/geographical-based ancestry group.

11. The computer-implemented method according to claim 1, further comprising, as a first preliminary step, a step of general validation or ancestry grouping model, comprising the following sub-steps:
performing said step of identifying an individual-specific genetic reference group of individuals for each of the individuals of said reference panel;
for each of the identified individual-specific genetic reference groups, evaluating the prediction accuracy of the respective tailored Polygenic Risk Score (PRSTi) and comparing the respective tailored Polygenic Risk Score (PRSTi) with prediction accuracy of a standard Polygenic Risk Score debiased from ancestry-related effects based on a labelling-based continental ancestry model or geographical ancestry model which assigns the individual to a predefined continental/geographical-based ancestry group;
validating as a usable ancestry grouping model a model providing better performance than the labelling-based continental ancestry model or geographical ancestry model.

12. The computer-implemented method according to claim 11, wherein the step of validating comprises:
counting a proportion of cases for which the neighborhood-based ancestry grouping model yields more accurate predictions;
counting a number of controls for whom the neighborhood-based ancestry grouping model yields more accurate predictions;
validating the neighborhood-based ancestry grouping model only if both the proportion of improved predictions in cases and the number of improved predictions in controls exceed a predefined threshold.

13. A computer-implemented method for predicting the risk of a disease for an individual, based on genetic information of the individual, comprising the steps of:
providing a dataset related to a reference panel including genetically characterized individuals with known disease status and diversified global ancestry;
identifying an individual-specific genetic reference group of individuals selected as a subset from said reference panel, wherein said step of identifying comprises:
computing a plurality of genetic distances of the individual from each of the individuals of the reference panel, wherein each genetic distance is a genetic distance of the individual from a respective one of the individuals of the reference panel;
selecting the individuals to be comprised in the individual-specific genetic reference group of individuals based on the computed genetic distances of the individual from all the individuals of the reference panel;
calculating a basic Polygenic Risk Score (PRS₀) of the individual with respect to a disease, to provide a risk prediction for said individual with respect to said disease;
determining a background PRS contribution due to ancestry based on said individual-specific genetic reference group of individuals;
debiasing the calculated basic Polygenic Risk Score (PRS₀) of the individual from ancestry-related effects, by removing the determined background PRS contribution due to ancestry, to obtain the tailored Polygenic Risk Score (PRS_{T}) specific to the individual, and provide an improved risk prediction for said individual with respect to said disease;
predicting the risk of the disease for the individual based on the calculated tailored Polygenic Risk Score (PRS_{T}) specific to the individual and debiased with from ancestry-related effects.

14. The computer-implemented method according to claim 1, further comprising, as a first preliminary step, a step of general validation or neighborhood-based ancestry grouping model, comprising the following sub-steps:
performing said step of identifying an individual-specific genetic reference group of individuals for each of the individuals of said reference panel;
for each of the identified individual-specific genetic reference groups, evaluating prediction accuracy of the respective tailored Polygenic Risk Score (PRS_{Ti}) and comparing the respective tailored Polygenic Risk Score (PRS_{Ti}) with the prediction accuracy of a standard Polygenic Risk Score debiased from ancestry-related effects on the basis of a labelling-based continental or geographical ancestry model which assigns the individual to a predefined continental/geographical-based ancestry group;
validating as a usable ancestry grouping model a model providing better performances than the labelling-based continental or geographical ancestry model.
